(19) United States
(12) Patent Application Publication
(10) Pub. No.: US 2021/0015743 A1
(43) Pub. Date: Jan. 21, 2021

(54) TREATMENT OF IRRITABLE BOWEL AND INFLAMMATORY BOWEL DISEASE

(71) Applicant: Yoseph Seyoum, Silver Spring, MD (US)

(72) Inventor: Yoseph Seyoum, Silver Spring, MD (US)

(21) Appl. No.: 16/931,128

(22) Filed: Jul. 16, 2020

Related U.S. Application Data
(60) Provisional application No. 62/874,764, filed on Jul. 16, 2019.

(51) Int. Cl. A61K 9/00; A61B 5/00; A61K 31/05; A61K 9/48

(52) U.S. Cl. CPC A61K 9/0053; A61B 5/4255; A61B 5/0022; A61B 5/7495; A61B 5/4842; A61K 31/05; A61K 9/4808; A61B 5/748

(57) ABSTRACT

The present invention provides compositions and methods of delivery of the compositions, as well as diagnostic method steps that greatly alleviate symptoms associated with IBS and/or IBD. Diagnostic procedures determine a likelihood of one or both of IBS and IBD within a patient. The methods disclosed act to determine relevant biometrics and a cycle time for a specific digestive tract transit time in order to determine a proper dosage amount and potency and a time of administration of the compositions to facilitate delivery of active agents to targeted areas of the GI tract.

### CROSS-REFERENCE TO RELATED APPLICATIONS

This application claims priority to U.S. Provisional Patent Application Ser. No. 62/874,764, filed on Jul. 16, 2019, and entitled Treatment of Irritable Bowel and Inflammatory Bowel Disease. The contents of this provisional application are relied upon and incorporated herein by reference.

### FIELD OF THE DISCLOSURE

The present disclosure relates to methods and compositions for the treatment of digestive tract maladies. More specifically, the present invention relates to administration of compositions containing cannabidiol and terpenes to specific areas of the digestive tract.

### BACKGROUND OF THE DISCLOSURE

Presently, Irritable Bowel Syndrome and Inflammatory Bowel Disease result in numerous healthcare visits and are responsible for considerable costs on a recurring basis. Essentially each is a Functional GI Disorder. Cannabinoids, such as tetrahydrocannabinol (“THC”), cannabidiol (“CBD”), and other constituents have been found to alleviate many if not all the symptoms of IBS in medical marijuana patients across the country

Cannabinoids work by stimulating receptors in the brain and immune system, and some other places in the body as well. These receptors are part of the human Endocannabinoid System, which can regulate homeostasis in the human body and/or a “neutral” position of stable equilibrium between interdependent systems the human body.

However, treatment of IBS and IBD with Cannabinoids have been limited because of inadequate diagnostics and delivery mechanisms. While it is easy to smoke or vaporize cannabis to deliver cannabinoids to the circulatory system, these actions are generally systemic and do not deliver the medicine directly to an area of the body where the medicinal effect is most needed.

### SUMMARY OF THE DISCLOSURE

Accordingly, the present invention provides compositions and methods of delivery of the compositions, as well as diagnostic method steps that greatly alleviate symptoms associated with IBS and/or IBD. According to the present invention, diagnostic procedures determine a likelihood of one or both of IBS and IBD and further act to determine a cycle time for a specific digestive tract in order to properly time administration of the compositions to targeted areas of the GI tract.

Compositions according to the present invention include CBD combined with selected terpenes and a carrier oil. Preferred embodiments include a blend of CBD combined with THC and other terpenes in a ratio that minimizes any psychoactive effects being experienced by a patient received the composition.

In some embodiments, a therapeutic treatment using one or more variants of timed-release capsules containing predetermined amounts, strains, or compositions of CBD or THC may be administered using a smart-device application. The smart-device application may prompt the user to input symptoms, specific formulation taken, time of taking medication, etc. to continuously assess a treatment plan.

In the following sections, detailed descriptions of examples and methods of the invention will be given. The description of both preferred and alternative examples, though thorough, are exemplary only, and it is understood that, to those skilled in the art, variations, modifications and alterations may be apparent. It is therefore to be understood that the examples do not limit the broadness of the aspects of the underlying invention as defined by the claims.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates block diagram of a delivery mechanism for delayed delivery of active agents included in a therapeutic combination administered to a patient via a digestive tract.

FIG. 2 illustrates an exemplary release timeline according to some embodiments of the present invention.

FIG. 3 illustrates exemplary alternative capsule for delivery of active agents to specified areas of a digestive tract.

FIG. 4 illustrates additional exemplary method steps that may be executed in some implementations of the present invention.

FIG. 5 illustrates an exemplary flow diagram for administering a therapeutic combination of cannabinoids facilitated by a smart-device application.

FIG. 6 illustrates an exemplary embodiment of an automated controller that may be used to implement various aspects of the present invention.

FIG. 7 illustrates a block diagram of an exemplary mobile device for use in connection with the present invention.

FIG. 8 illustrates an exemplary smart device displaying an interactive interface with a reminder screen as part of a smart-device app usable in connection or as part of the present invention.

FIG. 9 illustrates an exemplary smart device displaying an interactive interface with a graph screen as part of a smart-device app usable in connection or as part of the present invention.

FIG. 10 illustrates an exemplary smart device displaying an interactive interface with a symptom input screen as part of a smart-device app usable in connection or as part of the present invention.

FIG. 11 illustrates an exemplary smart device displaying an interactive interface with a detailed graph screen as part of a smart-device app usable in connection or as part of the present invention.

FIG. 12 illustrates an exemplary smart device displaying an interactive interface with a download screen as part of a smart-device app usable in connection or as part of the present invention.

FIG. 13 illustrates an exemplary smart device displaying an interactive interface with a scanning screen as part of a smart-device app usable in connection or as part of the present invention.

FIG. 14 illustrates an exemplary embodiment of user-tailored packaging in connection with the present invention.

FIG. 15 illustrates another exemplary flow diagram for administering a therapeutic combination of cannabinoids facilitated by a smart-device application.

### DETAILED DESCRIPTION

The present disclosure provides generally for compounds for delivery to disparate portions of a digestive tract of a mammalian patient and methods for determining a need for administration of the compositions. Some preferred embodiments additionally include methods for determining an efficacious timing interval between doses of the composition. Through this description, “patient” and “user” may be used interchangeably, especially with respect to the smart-device application described herein. However, in some embodiments, these may be two different individuals: the user may use the smart-device application to facilitate care of a patient. The interchangeable use of these two terms is not meant to limit the scope of this disclosure.

According to the present disclosure, a composition may include be a timed-release capsule taken 1-4 times daily to manage symptoms encountered by IBS and/or IBD patients, as well as symptoms of general stomach upset. In some exemplary embodiments, a composition according to the present invention releases cannabinoids and terpenes in two or more timed phases over a predetermined period of time (e.g. 8-10 hours) and at targeted areas of the patient's digestive tract. Targeted areas of the patient may include, one or more of: a stomach; a small intestine; and a colon, where symptoms of IBS are experienced typically experienced. A ratio of CBD to THC and the dosing of the THC itself may be tailored to minimize the occurrence of any psychoactive effects felt by the patient.

Some exemplary embodiments include a composition comprising a hard gelatin capsule containing a mixture of Medium Chain Triglyceride (MCT) Oil and cannabinoids and terpenes to targeted areas of a patient's body. Other carriers may also be used. A blend of cannabinoids and terpenes calms linings of the digestive tract and their surrounding musculature, resulting in fewer muscle spasms and an overall feeling of a calmed digestive tract leading to increased comfort for the patient.

Ratios of Cannabinoids and Terpenes combined according to the present invention work together in an “Entourage Effect”, popularized and studied by Dr. Ethan Russo. The Entourage Effect is a synergistic effect observed between the two types of compounds. According to the present invention, CBD is combined with THC and other terpenes in a composition with ratios and amounts of each constituent that are conducive to inducing an Entourage Effect. The ratios and amounts of each constituent administered in combination provides a more efficacious effect that administration of individual compounds alone.

In some exemplary embodiments, CBD and THC are included in an administered composition in a non-psychoactive ratio of 3:1 (CBD:THC), which has been shown to boost the immune system and alleviate gastrointestinal distress caused by functional GI disorders, as well as Crohn's and Colitis. Additionally, the composition may include cannabinoids to assist with one or more of: regulation of tissue inflammation; blocking of pain experienced, and reduction of pain experienced. Compositions may additionally include terpenes conducive to evoking a reduction in muscle spasms and nausea. Suitable Cannabinoids for a composition according to the present invention include, one or more of: CBD, THC, CBDa, THCa, THCV, and CBC.

A preferred composition may also include terpenes known to have helpful effects on regulating digestion and the sensation of pain. Terpenes are preferably dosed in small amounts, such as, for example amounts as small as 0.05% of a mixture. Compositions according to the present invention may include one or more of: β-Caryophyllene, α-Humulene, β-Myrcene, α-Bisabolol, Limonene, and Linalool or other terpenes that help the patient boost an immune function, decrease pain, decrease motility of bowel walls, increase absorption of cannabinoids, and generally calm the patients' gut.

In some embodiments, ratios of Cannabinoids and Terpenes may be adjusted to bring about an “Entourage Effect”, wherein an Entourage Effect is a synergistic effect observed between the two types of compounds that make one or more of the compounds be more effective in eliciting a desired effect from the patient's body as compared to just one type of compound alone.

In another aspects, terpene content in the composition may be different for a daytime formulation and a nighttime formulation to accommodate different schedules of a patient. Essentially, a daytime formulation will contain a relatively low ratio of THC and/or other psychoactive components and a nighttime formulation may include increased THC and/or other psychoactive components.

According to preferred embodiments of the present invention, botanically derived terpenes (terpenes from non-cannabis plants that share a same or similar chemical structure as cannabis-derived terpenes) are combined with a high-quality CO₂ solvent-less extraction of cannabinoids. Embodiments may include terpenes from cannabis plants in place of or in addition to botanically derived terpenes, however current regulatory and manufacturing constraints may make the botanically derived terpenes more readily available as preferred pharmaceutical grade components of the composition.

In some embodiments of the present invention, IBS may be viewed as a functional GI disorder which infers that an issue exists within the patient involving how the brain and GI tract work together. Typically, bowel muscles of a patient are diagnosed as moving and tightening in spasms. Additional diagnostic conditions may include a determination of the existence of one or more of: depression, anxiety, and somatic symptom disorder, where bodily pain or fatigue severely affects mental health. Physiological symptoms included in the diagnosis of IBS may include one or more of: diarrhea, constipation, pain, cramping, bloating, gas, and the sensation that the patient hasn't completed a bowel movement. Still other factors that may be considered in a diagnosis of IBS may include one or more of: a patient history of mental, emotional, and/or physical trauma, people under a lot of stress, and those with poor eating habits.

According to one aspect of the present invention, cannabis extract(s), such as, one or more of: CBD, THC and other terpenes are incorporated in a form factor that allows for the one or more of: CBD, THC and other terpenes to be released at different locations along a GI tract of a patient. The CBD, THC and other terpenes may be released at different locations along the GI tract via sustained release microspheres and/or time release capsules/pills when swallowed by patients delivers cannabis extract to small and large intestine for the treatment of irritable bowel syndrome and other gastrointestinal problems such as bloating, diarrhea, constipation, urgency, gas, pain at evacuation and a sense of an incomplete bowel movement. During some treatment methods of the present invention, the CBD, THC and other terpenes are delivered to a small or large intestine of the patient in a sustained and prolonged release effective to calm the gut muscles relieve pain and aid digestion.

Microencapsulation may include small particles or droplets surrounded by a coating thereby allowing capsule to pass beyond the stomach and into the intestinal tracts. According to the present invention, microencapsulation may enclose solids, liquids, or gases including CBD, THC and other terpenes inside a micrometric wall made of hard or soft soluble film. A microcapsule may include a small sphere with a uniform wall around it. Components with the microcapsule may be referred to as a core, internal phase, or fill, and the wall may be referred to as a shell, coating, or membrane. Some materials like lipids and polymers, such as alginate, may be used as a mixture to trap the material of interest inside. Microcapsules useful in embodiments of the present invention preferably include pores with diameters between a few micrometers and a few millimeters. Coating materials used for coating may include one or more of: ethyl cellulose; polyvinyl alcohol; gelatin; and sodium alginate.

Various embodiments include microcapsules of disparate shapes, such as: a crystal, a jagged adsorbent particle, an emulsion, a Pickering emulsion, a suspension of solids, or a suspension of smaller microcapsules. A microcapsule according to the present invention may have multiple walls.

In some embodiments, a therapeutic combination may provide a release from mere seconds to about 12 hours after on administration. Embodiments include release of more than about 90% or more of active agents in less than about 24 hours. In particular embodiments, an administration may provide a zero-order rate of release for at least a portion of the active agents during a 24-hour period following introduction into a GI tract of a patient. Some embodiments may additionally provide an ascending rate of release for at least a portion of the period of administration. Accordingly, an administration cycle may provide a relatively fast initial rate of release of active agents followed by a slower rate of release of the remaining active agents or an initial slow release of active agents followed by a subsequent relatively faster release of active agents.

In some embodiments, this therapeutic treatment may be administered by a smart-device application. The smart-device application may allow a patient to track cannabis strains, dosages, and formulations. It may also allow a patient to log the patient's symptoms or feeling of relief on a periodic basis, set reminders, and share data with other patients and doctors. In this way, a crowdsourced evaluation of appropriate strains, dosages, and formulations may be generated.

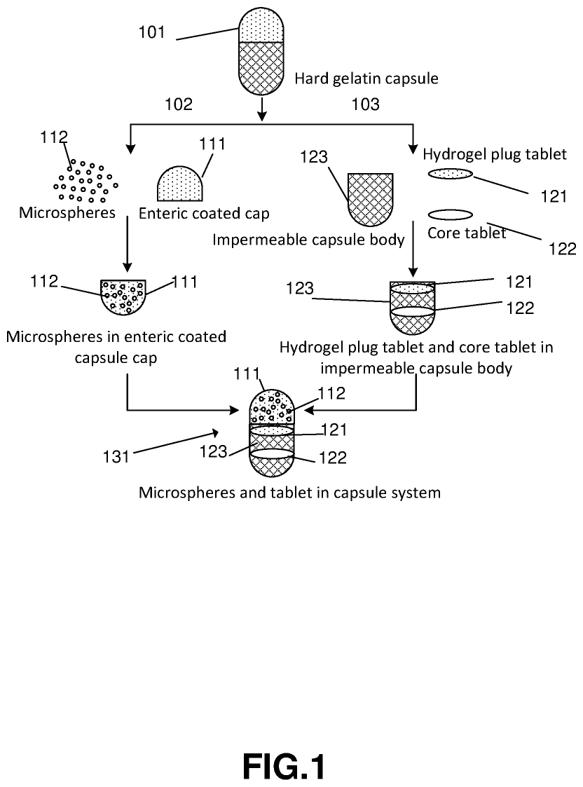

Referring now to FIG. 1, in some embodiments, a therapeutic combination may be incorporated into a hard gelatin capsule 101 as illustrated that enables multiple release stages. Although the capsule is illustrated with two release portions 102 103, with each portion releasable at a different time period following ingestion, other embodiments may include multiple time period releases.

Coasted release portion 102 may include an esoteric coated cap 111 with a coating that may dissolve based upon time after ingestion or a pH level around the cap. Microspheres 112 may lie within the coated cap 111 and be released upon dissolution of coated cap 111. Additionally, release portion 103 may include an impermeable capsule body 123 with a hydrogel plug tablet 121 that may temporarily block core tablet 122 until hydrogel plug tablet 121 is dissolved or removed. Microspheres 112 and core tablet 122 may contain medication, products, or other substances desirable for a delayed or staged delivery. (Cross-section of hard gelatin capsule 131 shows an exemplary arrangement of these constituent components within hard gelatin capsule 101.)

Additional time release mechanisms may be based on a pH level for GI tract site specific delivery of the therapeutic combination (an example of which include the mechanisms described by Hongjie Jiang, Wuyang Yu, Mark Oscai and Babak Ziaie, in “A Smart Capsule with a Hydrogel-Based pH-Triggered Release Switch for GI-Tract Site-Specific Drug Delivery,” IEEE Transactions on Biomedical Engineering, 2018, which is incorporated herein by reference). Other pH level-based mechanisms and/or other release timing mechanisms are also with the scope of the present invention. Still other release mechanisms suitable for release of cannabinoid formulations are described in U.S. Patent Application 2018/0263913 A1 to Lefler et al, which is incorporated herein by reference.

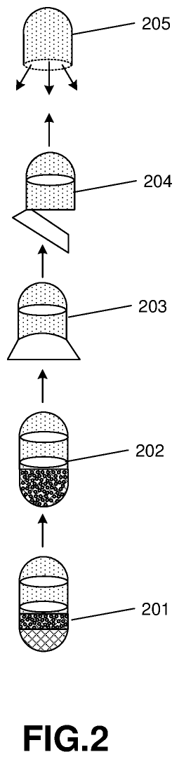

Referring now to FIG. 2, an exemplary sequence of release events for releasing the therapeutic combination is illustrated. At step 201 and time T1 a capsule is ingested into a GI tract of a mammal. A first lag time, such as 1-2 hours, follows ingestion with no drug release (some embodiments may include an initial drug release without delay in addition to those illustrated). At step 202 and time T2, active agents included in a therapeutic combination may release a first pulse of microspheres. The first release of active agents occurs at a first situs within the GI tract of the patient that ingested the capsule. T2 is preferably within 6-8 hours of T1.

At step 203, swelling of a plug tablet resulting from exposure to digestive fluid occurs. Typically, step 203 is not accompanied by any new active agent release, although some embodiments may include an active agent coated plug that allows for additional release of the active agent coating to be released during the time that the plug swells.

At step 204, a plug tablet may become dislodged from a remainder of the capsule. The dislodging of the plug tablet allows, at step 205, for a second release of active agents in the therapeutic combination at T3 (such as, for example, 10 hours after ingestion or after the first release). The second release of active agents in the therapeutic combination will typically be at a second situs within the GI tract of the patient. The difference in release situs may be estimated based upon a GI pass through rate for the patient and a respective dwell time (T2 and T3) prior to release of the active agents.

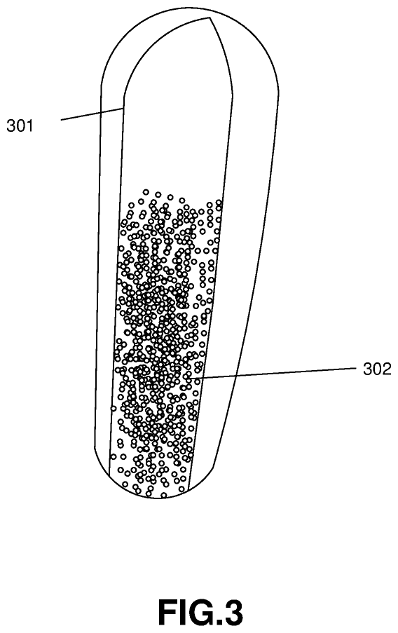

FIG. 3 illustrates a capsule 301 containing granules 302 of a therapeutic combination of active agents (as discussed above). The capsule 301 and/or the granules 302 may be comprised of a material designed to break down and/or dissolve at various disparate sites within a GI tract. Some examples of coatings that may be tailored to break down at different sites within the GI tract include esoteric coatings and enteric coatings. If the stomach area is targeted as a release situs for active agents, a capsule may be expandable, or include a mucoadhesive and/or bio-adhesive material.

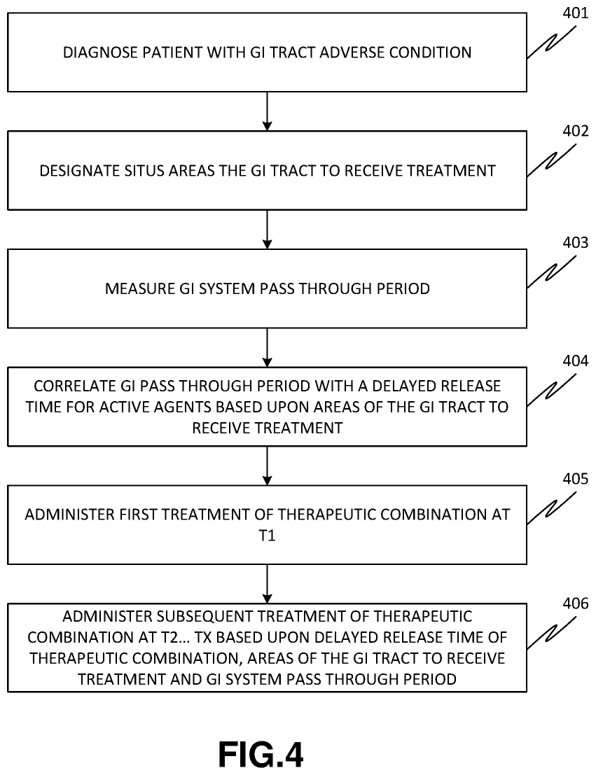

Referring now to FIG. 4, steps of a method that may be executed according to the present invention are illustrated. At step 401, a patient is diagnosed with a GI-adverse condition, such as one or both of IBS and IBD. Diagnosis may include quantification of patient symptoms, areas of discomfort, biometric measurements, ascertaining areas sensitive to pressure and other diagnosis steps.

At step 402, a health care practitioner, or the patient or other interested person, such as a pharmacist, may designate situs areas of the patient's GI tract to receive treatment with active agents included in a therapeutic combination (such as CBD, THC and terpenes discussed above).

At step 403, a GI Pass Through Period (sometimes referred to as GI Transit time) is measured. The GI Pass Through Period will include a period of time it takes for an ingested item to pass through the GI tract of the patient. Patient with GI disorders may have significantly different GI Pass Through Periods. For example, a patient with IBS may have a GI Pass Through Period of only 2-3 hours and a normal GI Pass Through Period may be 30 to 40 hours, depending upon a host of variables, such as food intake, emotional states of the patient and physical exertion of the patient. For the purposes of treatment according to the present invention, a GI Pass Through Period that estimates the conditions the patient will experience while ingesting the therapeutic combination is optimal.

At step 404, the GI Pass Through Period is correlated with a delayed release mechanism designed into a capsule containing the therapeutic combination. Correlation may include selection of a capsule with a particular dwell time for the therapeutic combination and/or different dwell times for different active agents.

At step 405, a first treatment of a therapeutic combination of active agents is administered at T1 and at step 406 subsequent treatment(s) of the same (or a different) therapeutic combination of active agents is administered at T2, . . . , Tx, wherein Tx is a total number of treatments at disparate times. According to the present invention, it is preferable to target two or more situs areas within a GI tract of a patient.

Table 1 illustrates an exemplary schedule of treatments and associated areas effected during each treatment as the therapeutic combination traverses the GI tract of the patient:

TABLE 1

| | Situs Area 1 | Situs Area 2 | Situs Area 3 |
|---|---|---|---|
| 1^{st} Dose | T1 | T2 | T3 |
| 2^{nd} Dose | T2 | T3 | T4 |
| 3^{rd} Dose | T3 | T4 | T5 |

As illustrated in Table 1, a timing (T2) of release of active agents in the therapeutic combination at Situs Area 2 is approximately the same as a time (T2) for administering a second dose of therapeutic combination of active agents. This pattern follows through Table 1 in order to illustrate the pendency of active agents within the GI tract at different situs. Other timing schedules are within the scope of the present invention.

The staggering of multiple treatment administrations (1^{st} dose-3^{rd} dose) results in simultaneous delivery of active agents to multiple situs throughout the GI tract of the patient as the treatment schedule progresses. In some embodiments, a specific dose of therapeutic combination, and/or active agents in each therapeutic combination administered may vary based upon one or more of: dosing sequence, body mass of the patient, frequency of administrations, GI transit time, and other variables.

In addition, some embodiments will include a specific therapeutic combination based upon a time of day of administration. For example, a T value associated with a nighttime of a patient may include administration of components conducive to pain relief and sleep, such as CBD oil, terpenes and THC, while a T value associated with a daytime experience will exclude THC or other psychoactive component.

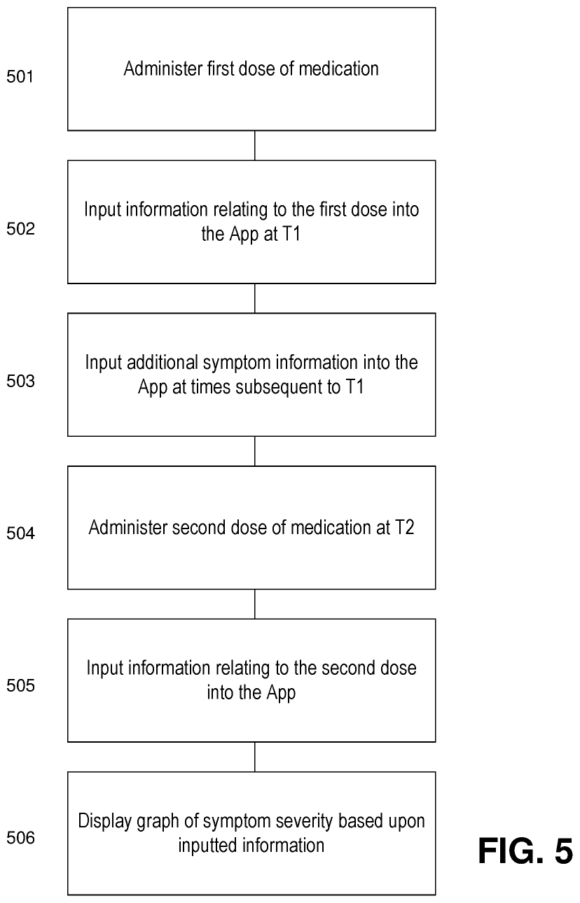

Referring now to FIG. 5, a method for administering cannabinoids to treat IBS (or similar symptoms) as facilitated by a smart device is shown. At step 501, a time designated as T1, a first dose of medication is administered. The medication may comprise any of the formulations described herein, with the accompanying cannabinoids, terpenes, or other additives. The first dose may comprise one or more strains or formulations, one or more ratios of constituent components (such as THC, CBD, etc.), one or more time-release stages, and one or more additives. The time-release stages may be determined with reference to the situs(es) to be treated; see Table 1 above. The additives may be any additive suitable to achieve a therapeutic effect. For example, some studies show that black pepper is a helpful additive to ameliorate some side effects of therapeutic administration of CBD. The medication may also comprise an ingestion technique. While the above disclosure discusses time-release capsules, in some embodiments, ingesting medication by smoking, vaporizing, or taking sublingual capsules or other pills may be appropriate.

At step 502, information relating to the first dose may be input into a smart-device application (the “App”). The App may comprise a human-readable interface on a smart device with one or more screens suitable for inputting information. Information that the user may input into the App includes, without limitation, the strains or formulations of the dose, ratios of constituent components, time-release stages, and additives. This information may be manually input by the user or obtained via a scan of a medication. For example, if the therapeutic formulation used as medication comes in a prescription bottle, then a bar code, QR code, or unique identifier visible on the bottle may be scanned to automatically populate information about the dose into the App. The user may be the same person as the patient or a different person, such as a medical practitioner, attendant, or family member.

Additionally, information about the symptoms experienced by the patient at T1 may be inputted into the App. This information may include, for example, a type of symptom, a severity of the symptom, an approximate duration of the symptom, and a numerical rating relevant to the symptom. For example, the user could input information about pain, diarrhea, bloating, or other IBS symptoms experienced by the patient.

In some embodiments, the user may also set a reminder. The reminder may comprise a duration of time or a specific time at which the App will automatically generate a reminder to take another dose of medication, update symptom information (such as duration and type), or provide other information useful in the facilitation of medical care.

At optional step 503, the user may input additional symptom information into the App at times subsequent to T1. The subsequent input(s) may be based on periodicity (e.g., inputting information every hour) or based on events (e.g., bowel movements or especially bad cramps). This step may be repeated one or more times before step 504.

At step 504, at time T2, a second dose is administered. The second dose may be substantially the same as the first dose, or it may modify one or more aspects of the first dose (such as strains, ratios, time-release stages, additives, or frequency of administration) of the first dose based on perceived changes in symptoms from the first dose. T2 may be based on the reminder optionally set at step 502. The App may provide one or more variations to the first dose based on the symptom information inputted at step 502. The second dose may also be based on preferences of the user; for example, if efficacy is similar, some users may prefer a higher quantity of THC at night (compared to CBD) and a higher quantity of CBD during the day. The second dose may also be based on efficacy information gathered from other users, such as other users of the App.

At step 505, information relating to the second dose may be inputted into the App via the human-readable interface, along with information relating to symptoms at T2. This information may be similar in character to the information input at T1 or at the optional periods of information input at step 503. This information may be updated subsequently based on periodicity or events, as at optional step 503.

At step 506, based on a user selection, a graph of symptom severity may be plotted against time. This graph may be supplemented with additional information, such as information relating to the dose ingested at times proximate to the data points on the graph. This graph may be based on information inputted by the user at symptom-input steps 502, 503, and 505 (as repeated as necessary).

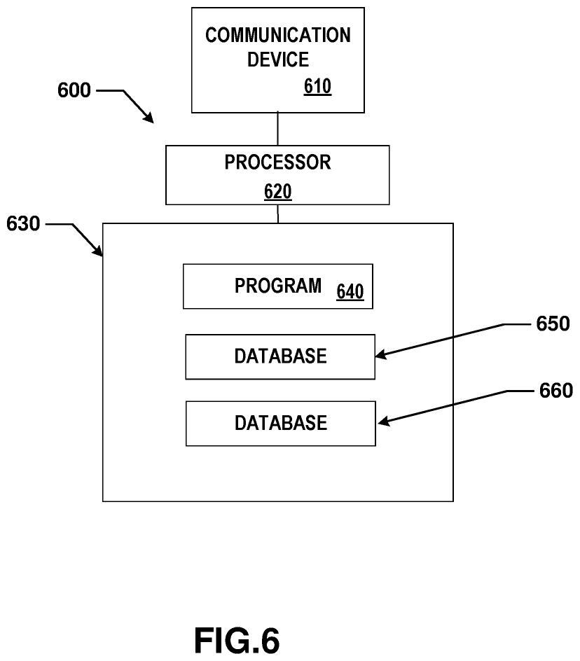

Referring now to FIG. 6, an automated controller is illustrated that may be used to implement various aspects of the present invention, in various embodiments, and for various aspects of the present invention. Controller 600 may be included in one or more of: a wireless tablet or handheld device, a server, a rack mounted processor unit, or a Smart Device. The controller may be included in one or more of the apparatus described above, such as a Server, and a Network Access Device. The controller 600 includes a processor 620, such as one or more semiconductor based processors, coupled to a communication device 610 configured to communicate via a communication network (not shown in FIG. 6). The communication device 610 may be used to communicate, for example, with one or more online devices, such as a personal computer, laptop, or a handheld device.

The processor 620 is also in communication with a storage device 630. The storage device 630 may comprise any appropriate information storage device, including combinations of magnetic storage devices (e.g., magnetic tape and hard disk drives), optical storage devices, and/or semiconductor memory devices such as Random Access Memory (RAM) devices and Read Only Memory (ROM) devices.

The storage device 630 can store a software program 640 with executable logic for controlling the processor 620. The processor 620 performs instructions of the software program 640, and thereby operates in accordance with the present invention. The processor 620 may also cause the communication device 610 to transmit information, including, in some instances, control commands to operate apparatus to implement the processes described above. The storage device 630 can additionally store related data in a database 650 and database 660, as needed.

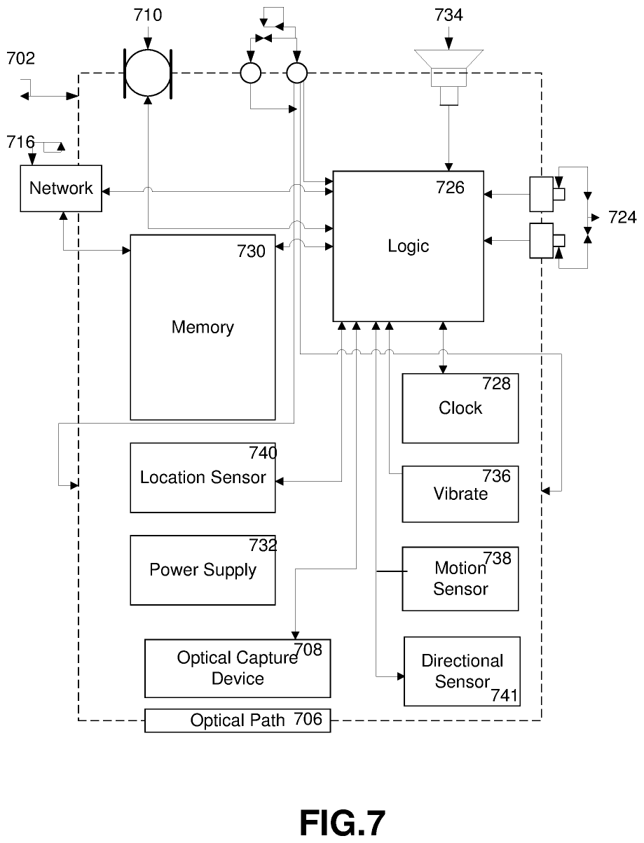

Referring now to FIG. 7, a block diagram of an exemplary mobile device 702 is shown. The mobile device 702 comprises an optical capture device 708 to capture an image and convert it to machine-compatible data, and an optical path 706, typically a lens, an aperture or an image conduit to convey the image from the rendered document to the optical capture device 708. The optical capture device 708 may incorporate a CCD, a Complementary Metal Oxide Semiconductor (known in the art as a “CMOS”) imaging device, or an optical sensor 724 of another type. Mobile device 702 may comprise a Smart Device.

A microphone 710 and associated circuitry may convert the sound of the environment, including spoken words, into machine-compatible signals. Input facilities may exist in the form of buttons, scroll wheels, or other tactile sensors such as touch-pads. In some embodiments, input facilities may include a touchscreen display.

Visual feedback to the user is possible through a visual display, touchscreen display, or indicator lights. Audible feedback 734 may come from a loudspeaker or other audio transducer. Tactile feedback may come from a vibrate module 736.

A motion sensor 738 and associated circuitry convert the motion of the mobile device 702 into machine-compatible signals. The motion sensor 738 may comprise an accelerometer that may be used to sense measurable physical acceleration, orientation, vibration, and other movements. In some embodiments, motion sensor 738 may include a gyroscope or other device to sense different motions.

A location sensor 740 and associated circuitry may be used to determine the location of the device. The location sensor 740 may detect GPS radio signals from satellites or may also use assisted GPS where the mobile device may use a cellular network to decrease the time necessary to determine location. In some embodiments, the location sensor 740 may use radio waves to determine the distance from known radio sources such as cellular towers to determine the location of the mobile device 702. In some embodiments these radio signals may be used in addition to GPS.

The mobile device 702 comprises logic 726 to interact with the various other components, possibly processing the received signals into different formats and/or interpretations. Logic 726 may be operable to read and write data and program instructions stored in associated storage or memory 730 such as RAM, ROM, flash, or other suitable memory. It may read a time signal from the clock unit 728. In some embodiments, the mobile device 702 may have an on-board power supply 732. In other embodiments, the mobile device 702 may be powered from a tethered connection to another device, such as a Universal Serial Bus (USB) connection.

The mobile device 702 also includes a network interface 716 to communicate data to a network and/or an associated computing device. Network interface 716 may provide two-way data communication. For example, network interface 716 may operate according to the internet protocol. As another example, network interface 716 may be a local area network (LAN) card allowing a data communication connection to a compatible LAN. As another example, network interface 716 may be a cellular antenna and associated circuitry which may allow the mobile device to communicate over standard wireless data communication networks. In some implementations, network interface 716 may include a Universal Serial Bus (USB) to supply power or transmit data. In some embodiments other wireless links may also be implemented.

As an example of one use of mobile device 702, a reader may scan some coded information from a location marker in a facility with the mobile device 702. The coded information may include for example a hash code, bar code, RFID, or other data storage device. In some embodiments, the scan may include a bit-mapped image via the optical capture device 708. Logic 726 causes the bit-mapped image to be stored in memory 730 with an associated time-stamp read from the clock unit 728. Logic 726 may also perform optical character recognition (known in the art as “OCR”) or other post-scan processing on the bit-mapped image to convert it to text. Logic 726 may optionally extract a signature from the image, for example by performing a convolution-like process to locate repeating occurrences of characters, symbols or objects, and determine the distance or number of other characters, symbols, or objects between these repeated elements. The reader may then upload the bit-mapped image (or text or other signature, if post-scan processing has been performed by logic 726) to an associated computer via network interface 716.

As an example of another use of mobile device 702, a reader may capture some text from an article as an audio file by using microphone 710 as an acoustic capture port. Logic 726 causes audio file to be stored in memory 730. Logic 726 may also perform voice recognition or other post-scan processing on the audio file to convert it to text. As above, the reader may then upload the audio file (or text produced by post-scan processing performed by logic 726) to an associated computer via network interface 716.

A directional sensor 741 may also be incorporated into the mobile device 702. The directional device may be a compass and be based upon a magnetic reading or based upon network settings.

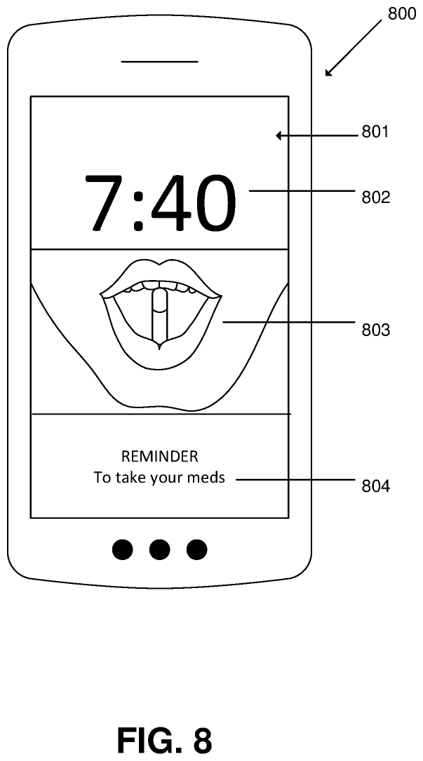

Referring now to FIG. 8, an exemplary smart device 800 displaying an interactive interface with a reminder screen 801 in an embodiment of the App is shown. Reminder screen 801 may assist a user or a patient in being made aware of appropriate times to take additional medication. The reminder time 802 may be based off patient input, data from a medical library, or other appropriate quantifier for appropriate times to take additional doses of medicine. Reminder screen 801 may also include a helpful visual graphic 803, which may provide instructions on taking medication, entertainment, medical literature, or other similar displays. Visual graphic 803 may be based upon user-inputted content or may be content received into smart device 800 from a communications device in logical connection with smart device 800. Reminder screen 801 may further include a helpful textual reminder 804, which may serve as a reminder, textual instructions, etc. related to the dose of medication occurring at reminder time 802.

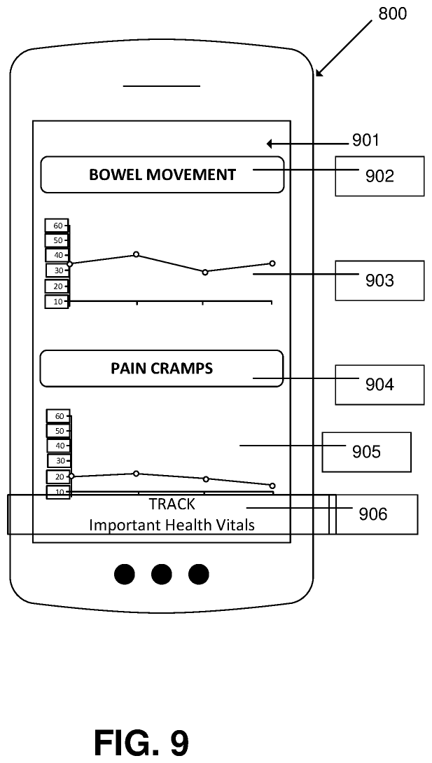

Referring now to FIG. 9, an exemplary smart device 800 displaying an interactive interface with a graph screen 901 of an embodiment of the App is shown. Graph screen 901 may display one or more graphs (shown here, as non-limiting examples, as bowel movement graph 903 and pain cramps graph 905) that may assist the user in tracking symptom trends over time, as well as one or more screen labels 906. The graphs may also use other independent variables (in addition to or instead of time) or other types of graphs as appropriate, such as histograms, bar graphs, scatterplots, etc.

As a nonlimiting example, graph screen 901 may include bowel movement graph 903. Bowel movement graph 903 may include graph title 902 and a plot on a pair of x-y axes. As shown here, bowel movement graph 903 uses time as an x axis (i.e., an independent variable). The y axis may be any suitable quantification associated with, in this case, bowel movements, such as a pain level associated with a bowel movement. Each data point may represent a time at which such quantification was logged (such as, for example, at steps 502, 503, and 505 of the embodiment shown in FIG. 5 and described in the accompanying text). Accordingly, the time component of each data point may be a literal time (e.g., a pain level of 40 was logged on Jul. 16, 2020 at 10:00 AM), a relative time (e.g., a pain level of 40 was logged 1 hour after the administration of the second dose), or other quantifiable time (e.g., a pain level of 40 was the second data point logged). Viewing this data over time may assist the patient in assessing the efficacy of a particular medication or therapeutic combination.

Similarly, graph screen 901 may also include pain cramps graph 905. Pain cramps graph 905 may include a title 904. Like bowel movement graph 903, pain cramps graph 905 may plot a quantification of a symptom against time on a pair of x-y axes based on symptom input data. In some embodiments, it may be useful to display both graphs simultaneously. This may be useful where the patient indicates multiple relief potentials (shown here as bowel movements and pain cramps) to assess the efficacy of a given medication (or other therapeutic combination) on each of the relief potentials.

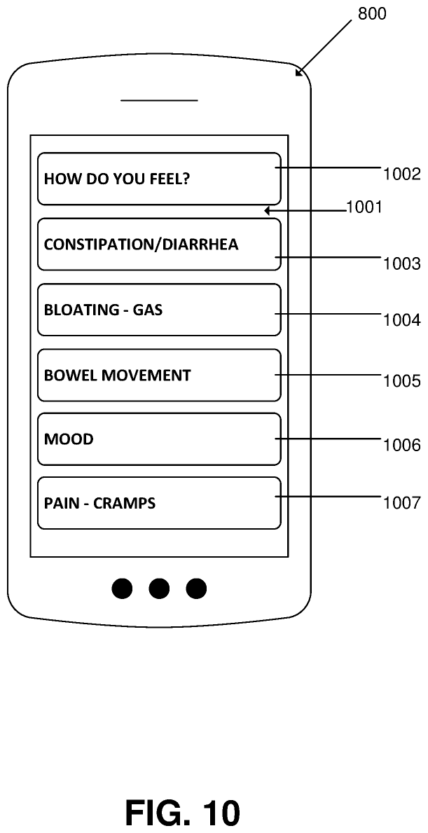

Referring now to FIG. 10, an exemplary smart device 800 with an interactive interface showing a symptom input screen 1001 in an embodiment of the App is shown. Symptom input screen 1001 may allow a patient or a user to input one or more symptoms, or one or more quantifications of symptoms related to chosen relief potentials, which may then form at least part of the dataset graphed on graph screen 901. Information inputted into symptom input screen 1001 may also be transmitted via a communication device to a central library, database, or other repository to give other users insights into the effect of various medications or other therapeutic combinations on various symptoms of the patient (or patients generally), which may be correlated with other information about the patient, such as body mass index, GI pass through time, etc. Symptom input screen 1001 may provide a prompt 1002 asking the patient how the patient feels from a physical or mental health perspective.

In some embodiments, symptom input screen 1001 may provide specific aspects about which to ask the patient with respect to the patient's health. These aspects may include specific relief potentials. For example, symptom input screen 1001 may provide a constipation/diarrhea prompt 1003. The patient may tap (or otherwise indicate a selection of) constipation/diarrhea prompt 1003. In some embodiments, such a selection may indicate that the patient is experiencing that particular symptom (here, constipation/diarrhea). In other embodiments, such a selection may cause the screen to navigate to a secondary screen asking the patient to input quantification information relating to the symptom (e.g., severity, amount, time of last occurrence, etc.). In some embodiments, the patient may create a custom relief potential for inclusion on symptom input screen 1001. Symptom input screen may also include bloating prompt 1004 (asking the patient about severity and frequency of bloating or gas), bowel movement prompt 1005 (asking the patient about severity and frequency of bowel movements, along with other medical indicators related to that, such as bleeding), and pain—cramps prompt 1007 (asking the patient about severity and frequency of pain due to cramps).

Symptom input screen 1001 may also ask the patient about a mood of the patient via mood prompt 1006. Mood prompt 1006 may ask about the patient's mood in any appropriate way, such as asking the patient to rate the patient's general mood on a scale (such as from 1-10) or by asking the patient to rate subsets of the patient's mood at the time of input, such as the patient's current happiness level, anger level, fatigue level, etc. In addition to providing information for graph screen 901 and for other users, this may assist the App in choosing other medications. For example, if the patient's mood does not respond well to a certain strain of CBD, then an alternative strain of CBD may be chosen.

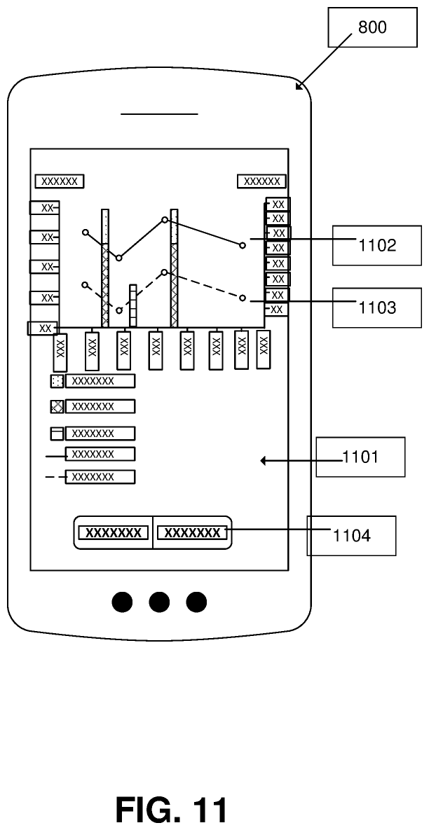

Referring now to FIG. 11, an interactive interface is illustrated with detailed graph screen 1101 shown on smart device 800 in an embodiment of the App. In some embodiments, detailed graph screen 1101 may allow the user to compare several symptoms (or other relief potentials) at once, or the same symptom tracked across multiple formulations of medication. As a non-limiting example, the first trendline 1102 may track datapoints across a first medication relating to the patient's pain over time. Second trendline 1103 may track datapoints across a second medication relating to the patient's pain over time. By presenting both graphs simultaneously, the patient may be able to best compare the efficacy of both medications on the patient's pain. Toggles 1104 may allow the user to view one medication at a time or may allow the user to toggle across multiple relief potentials. Detailed graph screen 1101 may also include bar graphs or graphs with multiple y axes (for example, as shown in FIG. 11, first trendline 1102 may track a first quantity of bowel movements, such as pain, and be plotted against the left-most y axis, while second trendline 1102 may track a second quantity of bowel movements, such as frequency, and be plotted against the right-most y axis).

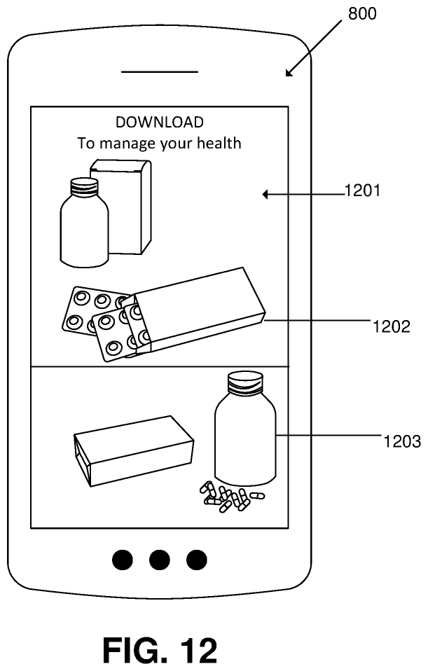

Referring now to FIG. 12, an exemplary download screen 1201 is shown on smart device 800. Download screen 1201 may display one or more medications 1202 1203. This may prompt the user to download additional information about medications 1202 1203 or to rate the user's experience with respect to those medications. Moreover, this screen may allow the user to order additional quantities of medications 1202 1203, to verify the appearance of medications 1202 1203 (such as by looking at codes inscribed on the medications), etc.

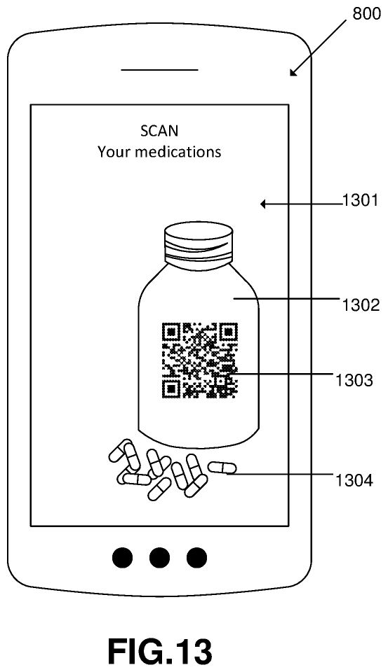

Referring now to FIG. 13, an interactive interface with a scanning screen 1301 is shown on a smart device 800. Scanning screen 1301 may display the output of an image capture device (such as a camera) affixed to or in communication with smart device 800. Scanning screen 1301 may allow a user to scan a medication 1302 to obtain more information about the medication, to order more quantities of the medication, or for easy input of medication details into the App for subsequent symptom monitoring and tracking.

In some embodiments, the image capture device may read off certain information from medication bottle 1302 (such as scanning for a name of the medication, a dosage, a CBD/THC ratio, etc.). In other embodiments, the image capture device may read QR code 1303 and use QR code 1303 to access an external database (such as via the internet) where the relevant details of the medication may be stored. In some embodiments, the image capture device may be able to scan individual capsules of the medication to look for distinctive characteristics (such as engraved codes) to identify the medication.

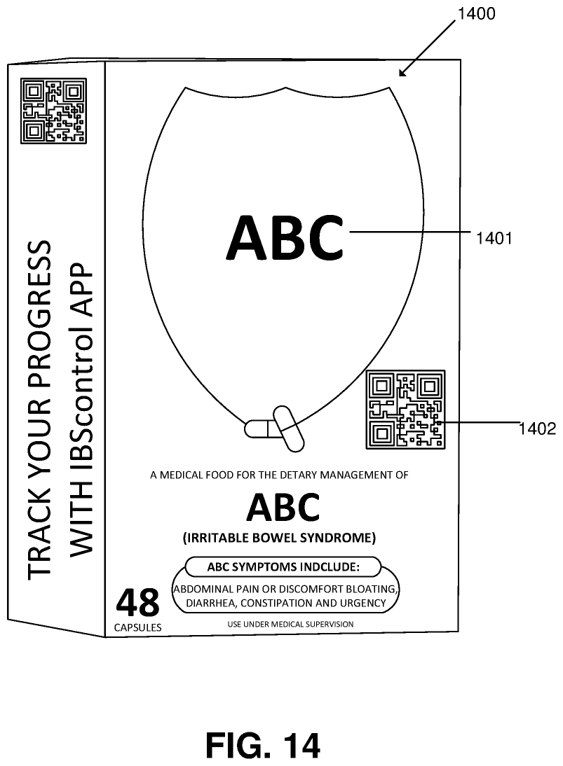

Referring now to FIG. 14, an exemplary embodiment of packaging 1400 for the cannabinoid capsules described herein is shown. Packaging 1400 may be customized for the specific type of Functional GI Disorder 1401 to be treated by the medications and methods described herein. An appropriate formulation (such as an appropriate ratio of THC to CBD) may be based on the Functional GI Disorder 1401. A QR code 1402 may be included to direct the purchaser to the App. QR code 1402 may be scanned (such as by smart device 800). In some embodiments, if smart device 800 is associated with a user (and the user's profile), then the scan may cause user-specific information to be transmitted to a database. This transmission may affect the version of the App to be downloaded to the smart device. For example, if the user is associated with a profile on the manufacturer's website, and the user has already indicated a medical preference (e.g., more THC at night), then the App may come pre-loaded with this preference and the user's profile already stored.

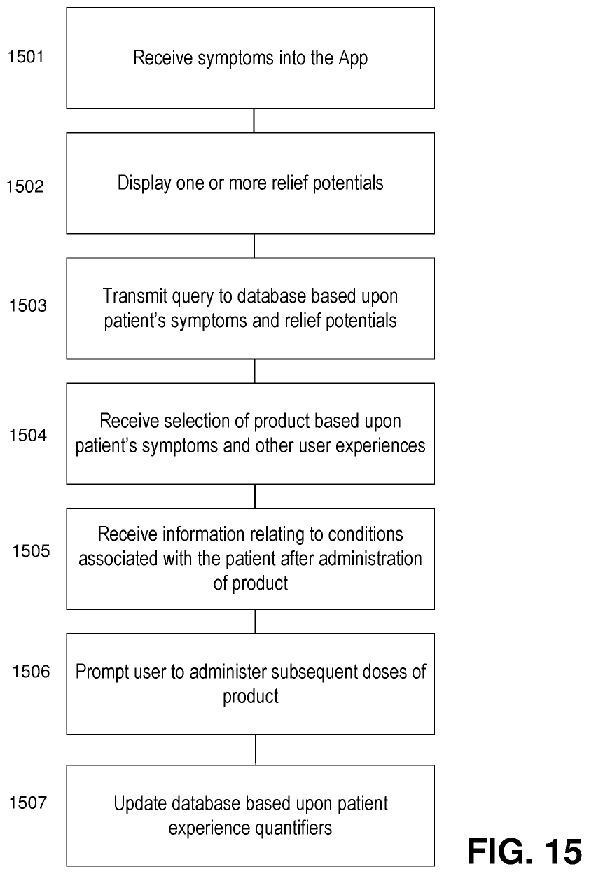

Referring now to FIG. 15, an alternative embodiment for therapeutic usage of cannabinoids to treat IBS, as facilitated by a smart-device application (the “App”), is shown. At step 1501, a user may be prompted by the App to input symptoms experienced by the patient. These symptoms may include any of various physiological manifestations of the state of the patient's body other than optimal health.

At step 1502, the App may provide one or more relief potentials for the patient. These relief potentials include, without limitation, alleviation of pain (especially pain associated with Functional GI Disorders), reduction of bloating, improvement of bowel control, improvement of digestion, and a reduction or elimination of bleeding. The user may select or prioritize any or all provide relief potentials.

Based on the inputted systems and chosen/prioritized relief potentials, at step 1503, the App may send a query to a database comprising input from other users based on those users' provided relief potentials and symptoms. Based on a return from that query, the App may recommend one or more products to assist the user in attaining the designated relief potentials. The products may include one or more strains of cannabinoids, one or more additives, or one or more release coatings, as described herein. These release coatings may comprise one or more concentric coatings. The concentric coatings may be around the same product (or other therapeutic compound) or around different products (thus allowing the products in the outer layers to release earlier in the process than the products contained in the inner layers).

In some embodiments, a number of disparate coatings may surround disparate products. This may be preferable where treatment involves the same or different products and additives in disparate coated portions. As discussed above, coating may be chosen to designate a release time or a desired situs along the GI track to target. As a result of the coating, a layer of the coating may dissolve under determined conditions, such as, without limitation, a number of hours after ingestion or a pH level of an area of the GI track. This may be particularly desirable where the patient has symptoms or designated relief potentials that may be mild: more potent medication may not be released during ingestion if the requisite pH level is not present. This may assist in product formulation and standardization. Accordingly, a time or situs of release of a product (or a portion, microencapsulation, or other subset thereof) may be determined by coating type and position in a concentric coating scheme.

Before, after, or contemporaneously with the display of the chosen product, the user interface may present other user experience aspects related to the product. For example, the user interface may display a plurality of products (to allow the user to choose based on one or more user preferences not expressed by the user to the application), coatings, or administration architecture of the product (e.g., whether the product comprises concentric coatings or otherwise disparate coating portions). The user interface may provide visuals or other educational material to allow the user to better understand the nature of the product being ingested by the user or patient. The user interface may also display comments from other users of the same (or similar) product, along with advice (from doctors or other users) about the product, such as other substances to ingest simultaneously with (or temporally proximate to) the product.

At step 1504, the user may select the product based upon the patient's symptoms and the experiences of other users. The App may display one or more products, along with any respective coatings, administration architecture, and other facts about the products, such as user ratings, user comments, side effects, other substances to ingest with the medication, or other useful information. The user may make the user's selection based at least in part upon the App's recommendation and then administer the product (or help a patient administer the product).

At step 1505, the user may input information relating to conditions associated with the patient. For example, the user may input symptom information, product information (which input may be obtained by scan of one or more products), and the timing of dosages taken by the patient. The user may input this at a time proximate to the time of administration of the product, along with at other times subsequent to the administration. For example, the user may input additional information associated with the patient periodically (e.g., hourly) or based upon the occurrence or non-occurrence of an event, such as a bowel movement.

At step 1506, the App may provide prompts for additional administration of the product at prescribed time intervals. In some embodiments, the App may alter its initial recommendations based upon subsequent queries to the databased based upon the patient's symptoms after administration of the first dosage. For example, if the first administration results in a reduction of cramping, and the user prioritized a reduction of cramping and an elimination of bleeding, then the App may recommend a product more efficacious for the elimination of bleeding.

The App may also prompt user input at designated time intervals for updated data, such as any of: symptom condition (including intensity, which may be rated quantitatively or qualitatively), patient activities (such as sleep, and the degree thereof; e.g., light, moderate, or heavy), physiological measurements (such as heart rate, body temperature, etc.), or other useful information. Based on this additional input, the App may suggest other medications or therapeutic treatments to the patient (or to other users of the App).

At step 1507, the App may update an internal library or a database based upon patient experience quantifiers. These quantifiers may include, without limitation, a use of the products, coatings (and measured responses thereto), time intervals, symptoms (whether measured quantitatively or qualitatively), and relief or result based upon the recommended product. In some embodiments, database queries by the user may prioritize previous inputs from the user to allow for the recommendation of products based upon products that have already worked for the user. In some embodiments, the App may also share patient outcomes with one or more other stakeholders, such as the patient's doctor. The App may also allow the patient to track the patient's progress, such as by graphs indicating symptoms or severity.

### CONCLUSION

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

In certain implementations, multitasking and parallel processing may be advantageous. Nevertheless, it will be understood that various modifications may be made without departing from the spirit and scope of the claimed disclosure.

What is claimed is:

1. A method for administration of a therapeutic combination to specific situs within a GI tract of a patient, the method comprising the steps of:
a. determining a GI system pass through period;
b. determining a body mass of the patient;
c. specifying two more situs within the GI tract of the patient for treatment with a first combination of therapeutic agents;
d. introducing a first combination of therapeutic agents comprising CBD and terpenes into the GI tract of the patient at a first time (T1), said first combination of therapeutic agents included in a capsule with a delayed release mechanism, said delayed release mechanism correlated to one or both of the two more situs within the GI tract of the patient specified for treatment; and
e. introducing a second combination of therapeutic agents comprising CBD and terpenes into the GI tract of the patient at a second time (T2), wherein T2 is based at least in part upon one of: the GI system pass through period and the body mass of the patient.

2. The method of claim 1 wherein the first combination of therapeutic agents comprising CBD and terpenes and the second combination of therapeutic agents comprising CBD and terpenes are equivalent.

3. The method of claim 1 wherein T2 comprises four or more hours.

4. The method of claim 1, further comprising the step of: inputting into a smart device, which smart devices comprises a processor, a memory, a communications device, a display capable of accepting user input, and software executable on command, first patient status information at T1.

5. The method of claim 4, further comprising the step of: inputting second patient status information at a time subsequent to T1.

6. The method of claim 5, further comprising the step of: inputting third patient status information at T2.

7. The method of claim 6, further comprising the step of: transmitting via the communications device one or more of: the first patient status information, the second patient status information, and the third patient status information to a database comprising one or more user experiences with one or more of: the first combination of therapeutic agents, the second combination of therapeutic agents, or a third combination of therapeutic agents.

8. The method of claim 7, further comprising the step of: displaying on the display a recommended fourth combination of therapeutic agents based upon data transmitted to the smart device via the communications device.

9. The method of claim 8, wherein the fourth combination of therapeutic agents comprises one or more of: a recommended product, a recommended coating, and a recommended administration architecture.

10. The method of claim 8, wherein the first patient status information comprises a severity rating of a symptom.

11. The method of claim 8, wherein the method further comprises the step of: prompting the patient to input one or more relief potentials.

12. An apparatus to assist in the administration of a therapeutic combination to a specific situs within a GI tract of a patient, the apparatus comprising:
a. a processor;
b. a memory;
c. a display;
d. a communications device;
e. software executable on command, the software operative to:
i. determine a GI system pass through period;
ii. determine a body mass of the patient;
iii. specify two more situs within the GI tract of the patient for treatment with a first combination of therapeutic agents;
iv. prompt the patient to introduce a first combination of therapeutic agents comprising CBD and terpenes into the GI tract of the patient at a first time (T1), said first combination of therapeutic agents included in a capsule with a delayed release mechanism, said delayed release mechanism correlated to one or both of the two more situs within the GI tract of the patient; and
v. prompt the patient to introduce a second combination of therapeutic agents comprising CBD and terpenes into the GI tract of the patient at a second time (T2), wherein T2 is based at least in part upon one of: the GI system pass through period and the body mass of the patient.

13. The apparatus of claim 12, wherein the software is further operative to prompt the patient to input first patient status information at T1.

14. The apparatus of claim 13, wherein the software is further operative to prompt the patient to input second patient information at a time subsequent to T1.

15. The apparatus of claim 14, wherein the software is further operative to prompt the patient to input third patient information at T2.

16. The apparatus of claim 15, wherein the software is further operative to cause the communication device to transmit one or more of: the first patient status information, the second patient status information, and the third patient status information to a database comprising one or more user experiences with one or more of: the first combination of therapeutic agents, the second combination of therapeutic agents, or a third combination of therapeutic agents.

17. The apparatus of claim 16, wherein the software is operative to display on the display a recommended fourth combination of therapeutic agents based upon data received into the communication device.

18. The apparatus of claim 17, wherein the fourth combination of therapeutic agents comprises one or more of: a recommended product, a recommended coating, and a recommended administration architecture.

19. The apparatus of claim 18, wherein the first patient status information comprises a severity rating of a symptom.

20. The apparatus of claim 18, wherein the software is further operative to prompt the patient to input one or more relief potentials.
